(19) United States
(12) Patent Application Publication
(10) Pub. No.: US 2004/0197889 A1
(43) Pub. Date: Oct. 7, 2004

(54) Recombinant expression of hhbv reverse transcriptase (rt)

(76) Inventors: Johannes Buchner, Ihlertstein (DE); Paul Muschler, Baar-Ebenhausen (DE); Martin Haslbeck, Wiesent (DE)

Correspondence Address: BAKER BOTTS LLP, C/O INTELLECTUAL PROPERTY DEPARTMENT, THE WARNER, SUITE 1300, 1299 PENNSYLVANIA AVE, NW, WASHINGTON, DC, 20004-2400, US

(21) Appl. No.: 10/476,056

(22) PCT Filed: Apr. 26, 2002
(86) PCT No.: PCT/EP02/04642

(30) Foreign Application Priority Data
Apr. 27, 2001 (EP) 01110073.2

(51) Int. Cl. C12N 9/22; C07H 21/04; C12N 15/86

(52) U.S. Cl. 435/199; 435/69.1; 435/456; 435/320.1; 435/325; 536/23.2

(57) ABSTRACT

The present invention relates to a method of producing a functional cell-free hepatitis B virus (HBV) reverse transcriptase (RT) comprising the steps of expressing HBV-RT in E. coli cells employing a suitable expression plasmid, or in a cell-free transcription-translation system and if expression was carried out in E. coli cells, lysing said E. coli cells and purifying HBV-RT from E. coli lysate or from said transcription-translation system. The invention further relates to a method of screening for an inhibitor of HBV-RT activity comprising the steps of contacting the cell-free HBV-RT produced according to methods of the present invention and with a potential inhibitor and assaying whether said potential inhibitor inhibits HBV-RT activity. Furthermore, the invention provides for a method of producing a pharmaceutical composition comprising the step of formulating the inhibitor identified by the screening method of the invention into a pharmaceutical composition.

The present invention relates to a method of producing a functional cell-free hepatitis B virus (HBV) reverse transcriptase (RT) comprising the steps of expressing HBV-RT in E. coli cells employing a suitable expression plasmid, or in a cell-free transcription-translation system and if expression was carried out in E. coli cells, lysing said E. coli cells and purifying HBV-RT from E. coli lysate or from said transcription-translation system. The invention further relates to a method of screening for an inhibitor of HBV-RT activity comprising the steps of contacting the cell-free HBV-RT produced according to methods of the present invention and with a potential inhibitor and assaying whether said potential inhibitor inhibits HBV-RT activity. Furthermore, the invention provides for a method of producing a pharmaceutical composition comprising the step of formulating the inhibitor identified by the screening method of the invention into a pharmaceutical composition.

Several documents are cited throughout the text of this specification. The disclosure content of each of the documents cited herein (including any manufacturer's specifications, instructions etc.) is hereby incorporated by reference.

Hepatitis B virus is a member of Hepadnaviridae, a family of small enveloped DNA viruses, which replicate genomes by reverse transcription of an RNA intermediate (Hu & Seeger, 1996). It causes acute and chronic human liver cirrhosis and hepatocellular carcinoma (Tavis & Ganem, 1993). The circular viral genome consists of 3221 bp (hHBV).

Hepadnaviral reverse transcriptase, the product of the pol gen, is expressed directly from the polycistronic viral (pregenomic) RNA (Hu & Seeger, 1996). Reverse transcriptase is a 95 kDa protein which is phosphorylated at several different sites (Ayola et al., 1993).

In the viral infection-cycle reverse transcriptase has to fulfill different tasks (Nassal, 1999). It is essential for packaging of viral RNA into the nucleocapsids (Hirsch et al., 1990), serves as a primer for minus strand DNA synthesis on the RNA-template (protein priming reaction) (Bartenschlager & Schaller, 1988; Wang & Seeger, 1992; Zoulim & Seeger, 1994), synthesizes the minus-strand DNA (reverse transcription) and subsequently the plus-strand DNA on the minus-strand DNA-template. In addition RT shows RNaseH activity towards RNA in RNA:DNA hybrids (Radziwill et al., 1990).

Recent work was focused on human (hHBV) and duck (dHBV) hepatitis (Hu & Seeger, 1996a). The assignment of functions to specific domains of the RT polypeptide was carried out using mutational analysis and sequence comparison. The four structural domains identified (Radziwill et al., 1990; Chang et al., 1990) comprise the N-terminal domain, a spacer region, the central reverse transcriptase domain and the C-terminal RNaseH domain.

The central reverse transcriptase domain and the C-terminal RNaseH domain show strong similarities to corresponding domains of other reverse transcriptases. The N-terminal domain (terminal protein, TP) is an unique feature of Hepadnaviridae which contains the priming tyrosin residue (hHBV: Tyr 63; Lanford et al., 1997). In this particular protein-priming process, the 5′-end of the minus-strand DNA is covalently linked to the reverse transcriptase and stays bound throughout the whole process of DNA synthesis (Bartenschlager & Schaller, 1988; Hu & Seeger, 1996).

The second process mediated by reverse transcriptase is the packaging of the pregenomic RNA together with the polymerase into virus particles. The initiation of nucleocapsid assembly is triggered by the ribonucleoprotein (RNP) complex and is essential for further reverse transcriptional activity (Hirsch et al., 1991). The RNP complex consists of polymerase (RT), an RNA sequence upstream to the pol gen and the so called ε-RNA or ε-sequence (Pollack & Ganem, 1994; Wang et al., 1994). The E-RNA folds into a defined stem loop which is recognized by the polymerase in cis. This means that the polymerase recognizes and binds its own mRNA, probably already during the process of translation. Downstream of the pol gen there is an additional ε-structure which seems not to be essential for the in vivo function of the polymerase (Lanford et al., 1997).

Besides the assembly of RNPs and the initiation of the packaging process, the ε-RNA exhibits two additional functions. One part of the stem-loop structure serves as a template (UUAC) for the synthesis of a 4 to 5 bp long primer by reverse transcriptase (Tavis et al., 1994). After synthesis of the covalently linked primer a so called template switch takes place. The primer detaches from the ε-RNA and binds to the proper transcriptional start region (UUAC) near the 3′end (DR1 element) of the pregenomic RNA (Tavis et al., 1994) resulting in the start of reverse transcription. An additional feature of ε-RNA in binding to reverse transcriptase is to induce a conformational change resulting in an enzymatically active protein. Reverse transcriptase in its active state is resistant towards proteases in vitro (Tavis and Ganem, 1996; Tavis et al., 1998).

Studies of reverse transcriptase expression in cell-free systems showed that host factors are essential for assembly of the RNP complex (Hu & Seeger, 1996). The activity of reverse transcriptase expressed in rabbit reticulocyte lysates was 50-times higher than that expressed in wheat germ extracts. It is known that wheat germ extracts do not contain a functionally equivalent Hsp90 (Dalman et al., 1989). This suggests that Hsp90 is an essential host component for reverse transcriptase activity. Co-immunoprecipitation experiments verified that Hsp90 binds to reverse transcriptase. The function of Hsp90 is probably to stabilize the newly synthesized reverse transcriptase in a conformation in which it is able to bind ε-RNA (Hu & Seeger, 1996b; Lanford et al., 1997). The Hsp90 complex interacting with reverse transcriptase contains p23 and hydrolyzes ATP in a manner known from the maturation of steroid hormone receptors (Hu et al., 1997; Pratt & Dittmar, 1998). Additional components of this complex are Hsp70 and probably Hsp40. The involvement of Hsp70 and p23 in the complex was shown by co-immunoprecipitation, antibody masking and RT activity assays. The ATP-dependence of the reaction can be inhibited by geldanamycin (Hu et al., 1997).

It is proposed that the chaperone-complex stays associated with reverse transcriptase during the viral replication cycle and is finally packaged into the nucleocapsid (Hu et al., 1997). This is in contrast to the transient interaction of Hsp90 with another natural substrate, the steroid hormone receptors (Pratt, 1993).

Mutagenesis studies revealed two Hsp90/p23 binding sites at reverse transcriptase, one in the TP region, the other one in the central polymerase domain. As for other Hsp90 substrates there is no conserved binding motif on reverse transcriptase. (Hu et al., 1997; Cho et al., 2000).

The DNA polymerase function of reverse transcriptase in virus particles was detected in 1973. RT isolated from virus particles was able to synthesize its own plus strand DNA. Exogenous templates like DNA or RNA strands were not accepted for DNA synthesis (Kaplan et al., 1973; Summers & Mason, 1982; Radziwill et al., 1988). Therefore, RT isolated from virus particles was not suitable for biochemical characterization.

Using in vitro translation systems, Wang and Seeger (1992) were able to express the dHBV RT in rabbit reticulocyte lysates for the first time. In 1993, Tavis and Ganem expressed an RT fusion protein in yeast cells. Expression of HBV-RT in Xenopus oocytes was done successfully by Seifer & Standring (1993). Landford et al. (1995) were able to express and purify hHBV-RT from insect cells using the baculovirus system.

RT isolated from the expression systems mentioned above showed the expected functions (reverse transcription, DNA polymerisation using RT-mRNA as template). In addition it was shown that isolated RT also used exogenous DNA and RNA templates (Tavis et al., 1998).

However these studies were performed in eukaryotic cell lysates which contain additional proteins potentially required for RT activity.

In recent years, new eukaryotic expression systems were developed and improved. In 1999, the yeast Ty1 expression system (Tavis & Ganem, 1993) was modified using a histidin marker to track HBV-RT activity (Qadri & Siddiqui). Ladner et al. (1997) developed an hepatoblastoma stem cell line in which the HBV-RT expression was regulated by tetracyclin concentrations in the medium.

In view of the above, the technical problem underlying the present invention was the efficient provision of functional HBV-RT that is essentially free of contaminating eukaryotic proteins or that can be easily purified from contaminating proteins.

The solution to said technical problem is achieved by providing the embodiments characterized in the claims.

Accordingly, the present invention relates to a method of producing a functional cell-free hepatitis B virus (HBV) reverse transcriptase (RT) comprising the steps of

(a) expressing HBV-RT in

(aa) E. coli cells employing a suitable expression vector; or

(ab) a cell-free transcription-translation system

(b) if expression was carried out in E. coli cells, lysing said E. coli cells and

(c) purifying HBV-RT from the E. coli lysate or from said transcription-translation system.

The term “functional” means, in accordance with the present invention, a HBV-RT polypeptide having reverse transcriptase activity. The activity can be analyzed by methods known in the art, including employing the RTA (Reverse Transcriptase Assay)-kit (RetroTech, Unterschleissheim, Germany). In general, present RT-assays make use of labeled nucleotides to be integrated into synthesized DNA, a process which is carried out by the reverse transcriptase. For quantification of reverse transcriptase activity in the in vitro systems, the incorporated (labeled) dUTP into DNA is measured, which acts as a parameter for RT activity. The measurements are usually based on signals caused by chemiluminescence, bioluminescence or radioactivity. Details of such a Reverse Transcriptase Assay are shown in example 4 of the present application.

The term “cell-free” as used according to the present invention denotes a system which is substantially free of life cells.

The term “hepatitis B virus (HBV) reverse transcriptase (RT)” as used herein relates to a polypeptide capable of the transcription of pre-genomic mRNA into nascent minus-strand DNA, the formation of plus strand DNA from nascent minus-strand DNA and completion of double-stranded DNA. HBV-RT can be encoded by the nucleic acid sequence comprised in the sequence deposited with GenBank accession number X51970.1 (http://www.ncbi.nlm.nih.gov; last update 25. 04. 2001). The corresponding wild-type protein sequence of the human HBV-RT has the GenBank accession number CAA36229 (http://www.ncbi.nlm.nih.gov; last update 25. 04. 2001).

The term “expression vector” as used herein encompasses a vector containing the HBV-RT polynucleotide. Said vector, according to the invention, is suitable for the expression of HBV-RT in E. coli cells. Thereby, the HBV-RT polypeptide can be recombinantly produced. The vector may be., for example, a phage, plasmid, viral, or retroviral vector. Retroviral vectors may be replication competent or replication defective. In the latter case, viral propagation generally will occur only in complementing host cells.

HBV-RT polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells. The HBV-RT polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include tetracycline, kanamycin or ampicillin resistance genes for culturing in E. coli. Appropriate culture mediums and conditions for the above-described E. coli cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Other suitable vectors will be readily apparent to the skilled artisan.

The term “E. coli” refers to the species Escherichia coli and is meant to comprise any useful strain suitable for the expression of foreign (poly)peptides. Suitable strains include DH10B, JM109, M15 [pREP4], BL21DE3, JM109 DE3, TOP10.

The term “transcription-translation system”, in the context of this invention, denotes to a cell-free expression system with coupled transcription and translation including the RTS 500 system (Roche, Switzerland). Briefly, the HBV-RT polynucleotide is cloned into an expression vector. In an in vitro reaction T7 RNA polymerase transcribes the template into mRNA, which is followed by translation of the ribosomal machinery present in the E. coli lysate. Thus, transcription and translation into the HBV-RT polypeptide take place simultaneously in the reaction compartment.

The term “lysing”, in accordance with the present invention, refers to a process conferred by techniques well-known in the art for opening cells by cell membrane-rupture and releasing the polypeptide. This process may e.g. be carried out by several cycles of freezing and thawing of said cells, by mechanical homogenizers, glass beads, by ultrasound sonication, French Press, or by using lysozyme. Preferably, for E. coli, cells are lysed by sonication followed by treatment with the French Press.

In accordance with the present invention the term “purifying” means that once expressed or synthesized, the polypeptide of the present invention can be purified according to standard procedures of the art, including ammonium sulfate precipitation, column chromatography, affinity chromatography, gel electrophoresis and the like; see, Scopes, “Protein Purification”, Springer-Verlag, N.Y. (1982). Substantially pure proteins, as a result of the purification process, of at least about 90 to 95% homogeneity are preferred, and 98 to 99% or more homogeneity are most preferred. Once purified, to the above recited degrees, the proteins may then be used therapeutically (including extracorporeally) or in developing and performing assay procedures.

Under some experimental conditions described in the examples it has been observed that a part of the HBV-RT produced in accordance with the present invention is degraded during the purification process. In order to avoid or reduce such degradation it is preferred that the expression system used employs an inducible promoter. It is further preferred that lysis and purification ensue 2 hrs after induction of expression. Additionally, the method of the invention envisages the use of protease inhibitors, wherein said protease inhibitors are/must be capable of being removed from purified HBV-RT during the last purification step, at the latest. Protease inhibitors are known in the art and include Pefabloc SC, Pefabloc SC Plus, PMSF, Complete™. They can be purchased by, e.g., Roche Diagnostics (Switzerland), SIGMA (Germany) and used in accordance with the manufacturers' instructions. For a less pronounced degradation, all steps of the lysis/purification protocol are preferentially conducted without interruptions at 4° C.

In accordance with the present invention, it could surprisingly be shown that the efficient production of functional HBV-RT can be effected without having to rely on the employment of disadvantageous eukaryotic cells or cell lines. As mentioned above, prior art studies were performed in eukaryotic cell lysates containing additional proteins potentially required for RT activity. In contrast to cellular eukaryotic systems, the present invention enables to eliminate interfering influences of such cellular components. It was surprisingly and unexpected that HBV-RT can be produced in an enzymatically active form in a prokaryotic host without further manipulation of the system. The present invention significantly facilitates further studies, e.g., of direct influences of inhibitors on HBV-RT activity.

In a preferred embodiment of the invention said HBV-RT is expressed as a fusion protein.

The term “fusion protein” relates to a (poly)peptide (i.e. a polypeptide or a peptide) comprising at least two amino acid sequences that are, under natural conditions, not fused to each other and are preferably heterologous to each other.

For example, the HBV-RT polypeptides can be fused to marker sequences, such as a peptide which facilitates purification of HBV-RT.

The marker amino acid sequence can be a hexa-histidine peptide, such as the tag provided in a pQE vector (QIAGEN, Inc., 9259 Eton Avenue, Chatsworth, Calif., 91311), among others, many of which are commercially available. As described in Gentz, Proc. Natl. Acad. Sci. USA 86 (1989); 821-824, for instance, hexa-histidine provides for convenient purification of the fusion protein. Another peptide tag useful for purification, the “HA” tag, corresponds to an epitope derived from the influenza hemagglutinin protein; see Wilson, Cell 37 (1984); 767.

It is a particularly preferred embodiment in accordance with the invention that said tag is selected, but not limited to, from the group consisting of His-tag, Streptavidin-tag, HA-tag, GST-tag, CBP-tag, MBP-tag, FLAG-tag, myc as well as single-chain fragments (sc Fvs) of antibody binding regions.

In a further particularly preferred embodiment said tag is fused N-terminally to said HBV-RT or C-terminally to said HBV-RT.

Moreover, the HBV-RT polypeptide, when fused to a second protein, can be used as an antigenic tag. Antibodies raised against the HBV-RT polypeptide can be used to indirectly detect the second protein by binding to the HBV-RT. Moreover, because secreted proteins target cellular locations based on trafficking signals, the HBV-RT polypeptides can be used as a targeting molecule once fused to other proteins. Examples of domains that can be fused to HBV-RT polypeptides include not only heterologous signal sequences, but also other heterologous functional regions. The fusion does not necessarily need to be direct, but may occur through linker sequences.

Moreover, fusion proteins may also be engineered to improve characteristics of the HBV-RT polypeptide. For instance, a region of additional amino acids, particularly charged amino acids, may be added to the N-terminus of the HBV-RT polypeptide to improve stability and persistence during purification from the host cell or subsequent handling and storage. Also, peptide moieties may be added to the HBV-RT polypeptide to facilitate purification. Such regions may be removed prior to final preparation of the HBV-RT polypeptide. The addition of peptide moieties to facilitate handling of polypeptides are familiar and routine techniques in the art.

Furthermore, HBV-RT polypeptides, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins; see, e.g., EP-A3 0 094 827; Traunecker, Nature 331 (1988); 84-86.

In many cases, the Fc part in a fusion protein is beneficial in therapy and diagnosis, and thus can result in, for example, improved pharmacokinetic properties; see, e.g., EP-A 0 232 262. Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5; see Bennett, J. Molecular Recognition 8 (1995), 52-58; Johanson, J. Biol. Chem. 270 (1995), 9459-9471.

In a further preferred embodiment step (c) of the method of the invention comprises the steps of

(ca) centrifugation of the lysate or the transcription-translation system at about 30000×g so as to remove the non-soluble fraction of the lysate or the transcription-translation system; and

(cb) removing the supernatant containing the HBV-RT.

By applying a sustained centrifugal force according to the present invention a fast concentration and first purification step of HBV-RT in suspension can be achieved. During centrifugation denser particles, e.g., from cell debris or non-soluble compounds from the transcription-translation reaction migrate toward the periphery of the centrifugation tube, and the HBV-RT containing supernatant can easily be removed.

In a further particularly preferred embodiment the method of the invention further comprises the step of (cc) purifying the supernatant via chromatography.

In an additional particularly preferred embodiment of the invention said chromatography is affinity chromatography wherein said affinity is specific for the tag.

In another preferred embodiment of the invention said HBV-RT is human HBV-RT (hHBV-RT).

The invention further relates to a method of screening for an inhibitor of HBV-RT activity comprising the steps of

(a) contacting the cell-free HBV-RT produced according to the method of the present invention with a potential inhibitor; and

(b) assaying whether said potential inhibitor inhibits HBV-RT activity.

The polypeptide of the present invention provides a basis for the development of a screening method for compounds that may be inhibitors of HBV-RT or their encoding genes. The term “inhibitor” or antagonist as used herein is meant to refer to an agent that downregulates (e.g., suppresses or inhibits) bioactivity of the HBV-RT protein. An inhibitor or antagonist can be a compound which inhibits or decreases the interaction between a protein and another molecule.

An inhibitor or antagonist can also be a compound that down-regulates expression of the HBV-RT gene or which reduces the amount of the wild-type protein present.

It will be appreciated that the present invention also provides screening methods (in accordance with the above) that allow a high-throughput-screening (HTS) of compounds that may be candidates for such inhibitors. For example, a candidate inhibitor or antagonist not known to be capable of binding to a HBV-RT polypeptide encoded by a HBV gene can be tested by contacting said polypeptide with a candidate inhibitor or antagonist under conditions permitting binding of ligands known to bind thereto, detecting the presence of any bound ligand, and thereby determining whether such candidate antagonist or inhibitor inhibits the binding of a ligand to said polypeptide.

Said inhibitor or antagonist may be chemically synthesized or microbiologically produced and/or comprised in e.g., cell extracts from e.g., plants, animals, microorganism. Furthermore, said compound(s) may be known in the art but hitherto not known to be capable of suppressing or inhibiting said polypeptide.

A candidate antagonist or inhibitor not known to be capable of binding to the HBV-RT polypeptide can be tested to bind thereto comprising contacting said polypeptide with a candidate antagonist or inhibitor under conditions permitting binding of ligands known to bind thereto, detecting the presence of any bound ligand, and thereby determining whether such candidate antagonist or inhibitor inhibits the binding of a ligand to a polypeptide as described above.

For example, proteins that bind to a polypeptide and might inhibit or counteract to said polypeptide can be “captured” using the yeast two-hybrid system (Fields, Nature 340 (1989), 245-246). A modified version of the yeast two-hybrid system has been described by Roger Brent and his colleagues (Gyuris, Cell 75 (1993), 791-803; Zervos, Cell 72 (1993), 223-232). Briefly, a domain of the polypeptide is used as bait for binding compounds. Positives are then selected by their ability to grow on plates lacking leucine, and then further tested for their ability to turn blue on plates with X-gal, as previously described in great detail (Gyuris, supra; WO 95/31544).

Another assay which can be performed to identify inhibitors and antagonists involves the use of combinatorial chemistry to produce random peptides which then can be screened for counteracting or inhibiting effects.

One such assay has recently been performed using random peptides expressed on the surface of a bacteriophage (Wu (1996), Nature Biotechnology 14, 429-431).

Another well-known in the art approach to rapidly identify inhibitors and antagonists of a polypeptide makes use of the BIACORE-system (Pharmacia, Sweden).

Further protocols for protein-protein interactions and for the identification of inhibitors are given, e.g., in Phizicky and Fields (Microbiological reviews, 1995). Such a screening method for an inhibitor according to the present invention may make use of, but is not limited to the use of, template RNA, Primer (e.g. oligo dT), dNTPs, radioactive labeled dUTP, or, template RNA, Primer (e.g. oligo dT), dNTPs, biotinylated dUTP, a marker enzyme (e.g. streptavidin coupled peroxidase), a substrate for the marker enzyme (e.g. AEBTS) and may contain a candidate inhibitor or antagonist, or a plurality of putative inhibitors or antagonists in order to identify the compound capable of binding to and inhibiting or antagonizing the activity of the translated protein.

In a preferred embodiment the method of the invention the assaying in step (b) is effected via a readout system.

The term “readout system” in context with the present invention means any substrate that can be monitored, for example due to enzymatically induced changes. Such read out systems are well known to those skilled in the art and comprise radioactive or non-radioactive assays. Detection methods in accordance with the present invention comprise measuring the detectable signal by calorimetric changes, bioluminescence, fluorescence, phosphorescence or radioactive label. Therefore, scintillation detector, spectrophotometer or a microtiter plate (ELISA) reader is applied, when appropriate.

Such a “readout system” and quantification system can make use of the ability of HBV reverse transcriptase activity to synthesize DNA integrating radiolabeled nucleotides, digoxigenin- or biotin-labeled nucleotides. For example, in an RT-assay purchased by Roche (Switzerland) the biotin-labeled DNA is bound to the surface of streptavidin-coated microtiter plate. Then an antibody to digoxigenin, conjugated to peroxidase is added and binds to the digoxigenin labeled nucleotides. In the final step, the peroxidase substrate is added. The peroxidase enzyme catalyzes the cleavage of the substrate to produce a colored reaction product. The absorbance of the samples is determined using a microtiter plate (ELISA) reader and directly correlates to the level of RT activity in the sample.

In a particularly preferred embodiment said readout system is a non-radioactivity-based assay, as depicted in example 4.

In a further preferred embodiment the assaying step (b) is qualitative or quantitative.

In an additional preferred embodiment of the invention the potential inhibitor is a plant product or a plant-derived product.

Plant products or a plant-derived products in the context of the present invention includes aqueous, alcoholic or lipophilic extracts of said plants potentially having an HBV-RT inhibiting activity. Suitable extraction, distillation, precipitation, filtration and/or chromatography procedures are well-known for the person skilled in the art.

In a particularly preferred embodiment of the invention said plant is Phyllantus.

Phyllantus according to the present invention is selected from a member of the Phyllantus family consisting of Phyllantus amarus, Phyllantus niruri, Phyllantus emblica, Phyllantus urinaria, Phyllantus myrtifolius Moon, Phyllantus maderas pratensis and Phyllantus ussuriensis.

In an additional particularly preferred embodiment said Phyllanthus is Phyllantus amarus.

According to the invention it is possible to use leaves, bark, flowers, seeds, fruits, stalks, branches, trunk, root and/or wood of Phyllanthus, preferably the herb, that is to say all above-ground parts of the plant. Moreover, it is possible for Phyllanthus to be used in comminuted form and/or in unmodified form, that is to say as whole leaf, as granules, powder, precipitate, extract, dried extract and/or exudate, with extracts or dried extracts being preferred.

In addition, the invention relates to a method of refining the inhibitor identified by the method of the invention comprising (a) modeling said inhibitor by peptidomimetics and (b) chemically synthesizing the modeled inhibitor. A most suitable starting point for modeling by peptidomimetics is to test libraries of peptides of different lengths and sequences for inhibition of HBV-RT.

The invention further relates to a method of modifying an inhibitor identified by the method of the invention as a lead compound to achieve (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetales, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiozolidines or combinations thereof.

The various steps recited above are generally known in the art. They include or rely on quantitative structure-action relationship (QSAR) analyses (Kubinyi, 1993), combinatorial biochemistry, classical chemistry and others (see, for example, Holzgrabe and Bechtold, 2000).

Moreover, the present invention relates to a method of producing a pharmaceutical composition comprising the step of formulating the inhibitor identified by the screening method or refined according to the method as defined herein above with a pharmaceutically acceptable carrier and/or diluent.

The term “pharmaceutical composition”, as used in accordance with the present invention, comprises at least the inhibitor as identified herein above, such as a protein, an antigenic fragment of said protein, a fusion protein, a nucleic acid molecule and/or an antibody as described above and, optionally, further molecules, either alone or in combination, e.g., molecules which are capable of optimizing antigen processing, cytokines, immunoglobulins, or lymphokines, optionally, adjuvants.

The therapeutically useful compounds identified according to the invention may be administered to a patient by any appropriate method for the particular compound, e.g., orally, intravenously, parenterally, transdermally, transmucosally, or by surgery or implantation (e.g., with the compound being in the form of a solid or semi-solid biologically compatible and resorbable matrix) at or near the site where the effect of the compound is desired. Therapeutic doses are determined to be appropriate by one skilled in the art.

The pharmaceutical compositions can also include, depending on the formulation desired, pharmaceutically acceptable, usually sterile, non-toxic carriers or diluents, which are defined as vehicles commonly used to formulate pharmaceutical compositions for animal or human administration. The diluent is selected so as not to affect the biological activity of the combination. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. A therapeutically effective dose refers to that amount of inhibitor which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

Further examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected as stated above. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment.

The compositions comprising, e.g., the inhibitor which is a polynucleotide, polypeptide, antibody, compound drug, pro-drug or pharmaceutically acceptable salts thereof may conveniently be administered by any of the routes conventionally used for drug administration, for instance, orally, topically, parenterally or by inhalation. Acceptable salts comprise acetate, methylester, HCl, sulfate, chloride and the like. The drugs may be administered in conventional dosage forms prepared by combining the drugs with standard pharmaceutical carriers according to conventional procedures. The drugs and pro-drugs identified and obtained in accordance with the present invention may also be administered in conventional dosages in combination with a known, second therapeutically active compound. Such therapeutically active compounds comprise, for example, those mentioned above. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Examplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampule or nonaqueaous liquid suspension.

Moreover, the present invention relates to an in vitro method of synthesizing a desired cDNA using the HBV-RT produced in accordance with the method of the invention.

Finally, the present invention relates to a method of evaluating the batch to batch consistency of Phyllantus amarus extracts/inhibitor preparations and/or their shelf life, comprising contacting the HBV-RT produced in accordance with the invention with said batch and assessing the RT-activity. Said batches may be stored at −20° C., with or without repeated cycles of freezing and thawing, at 4° C., or at room temperature (22° C.) for e.g. 1 h, 24 hrs, 1 month, or 6 months.

The test may be carried out as described for instance in example 4 with the following modifications: Instead of HIV-RT, HBV-RT produced in accordance with the method of the invention is used in the Reverse Transcriptase Assay. If applicable, an internal standard may be included.

The figures show:

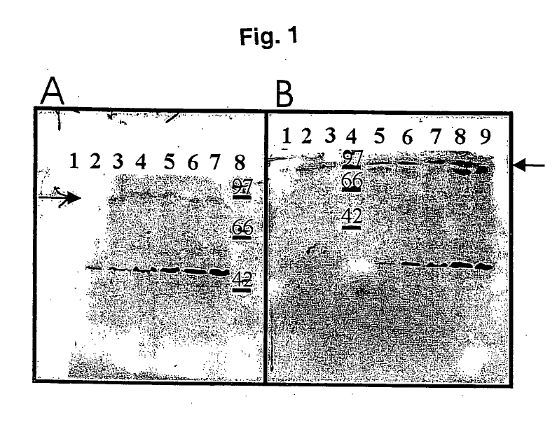

FIG. 1:

Detection of hHBV-RT Expressed in E. coli (Western Blot)

The figure shows the expression kinetics of hHBV RT in E. coli. Using the pBAD expression system the RT protein was detectable 1 h after induction with 11 mM IPTG. Besides the RT band (ca. 95 kDa) proteolytic fragments of RT could also be visualised (FIG. 1A.). RT and its proteolytic fragments were in the soluble fraction. The pellet fraction (inclusion bodies) did not contain RT (data not shown). The maximum amount of native RT appeared about 2 h after induction. 3 h after induction the amount of native RT decreased whereas the amount of proteolytic RT-fragments (ca. 50 kDa band) still increased 5 h after induction.

In E. coli cells, containing the pIVEX-expression-system (FIG. 1B.), native RT is already expressed at low levels before induction. In contrast to the pBAD-system, the pIVEX-expression-system is not tightly repressed. The maximum amount of native RT was observed ca. 3 to 4 h after induction.

For negative controls (1) M15 pREP4 cells carrying the pQE::hsp82-vector, (2) BL21DE3 cells with pIVEX and (3) M15 pREP4 cells carrying the pQE30::hsp82 vector were used. Lysates of these cells induced with IPTG or arabinose were separated on SDS-Page and subsequently blotted. No bands were detected in the range of 97 kDa (data not shown). This shows that only in cells containing the plasmids pIVEX::RT and pBAD::RT the hHBV-RT was expressed.

FIG. 1(A) lanes 1 to 8 contain the following fractions: [1] bis [7]: Cell-lysates at time points t=1 h before induction, at the time point of induction t=0 and 1 h, 2 h, 3 h, 4 h and 5 h after induction.; [8] LMW-protein standard (Biorad).

FIG. 1(B) lanes 1 to 9 contain the following fractions: [1] to [3]. Cell-lysates at time points t=4 h, 3 h and 1 h before induction; [4] LMW-protein-standard (Biorad); [5] to [9]: Cell-lysate at time of induction and 1 h, 2 h, 3 h und 4 h after induction. Gels used for blotting contained 10% Polyacrylamid.

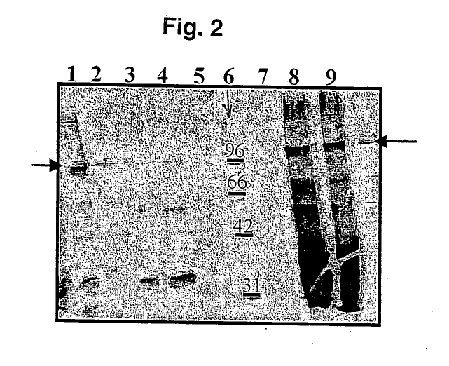

FIG. 2:

The figure shows the Western blot of RT-purification from E. coli lysates. Using αRT-antibodies Reverse Transcriptase was detected in the cell lysate and the eluted fractions. Wash fractions contained only small amounts of RT. Besides the band for native RT (ca. 95 kDa) proteolytic fragments have been detected. No RT was detected in the pellet fractions (data not shown). As a result, RT from hHBV was successfully expressed in E. coli.

Lanes 1 to 8 contain the following fractions:

[1]: Cell-lysate 5 h after induction; [2] to [5]: Wash fractions; [6] LMW-protein-standard (Biorad); [7] to [9] Eluted fractions. Proteins were separated using SDS-Page (10% polyacrylamid). Arrows indicate the position of native RT.

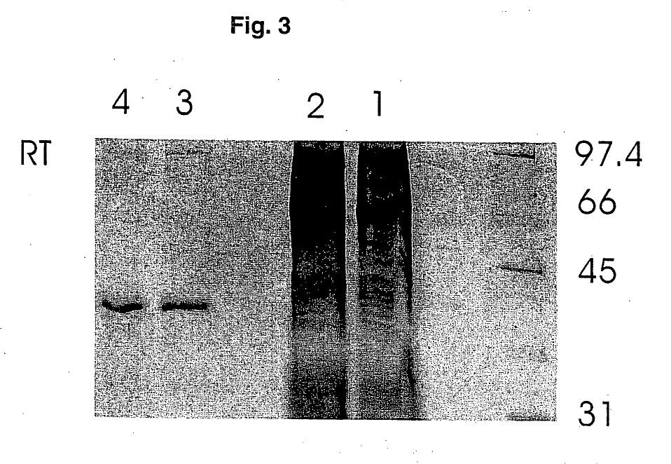

FIG. 3:

FIG. 3 shows a SDS-PAGE of hHBV-RT protein samples using a T7-vector system expressing RT from using an in-vitro transcription/translation system.

Lane 1: reaction after 20 h expression at 30° C.; 15 μg of T7-expression plasmid with N-terminal His-tag; lane 2: reaction after 20 h expression at 30° C.; 15 μg of T7-expression plasmid with used N-terminal Strep-tag; lane 3: RT purified from reaction lane 1 according to His-tag purification protocol; lane 4: RT purified from reaction lane 2 according to Strep-tag purification protocol.

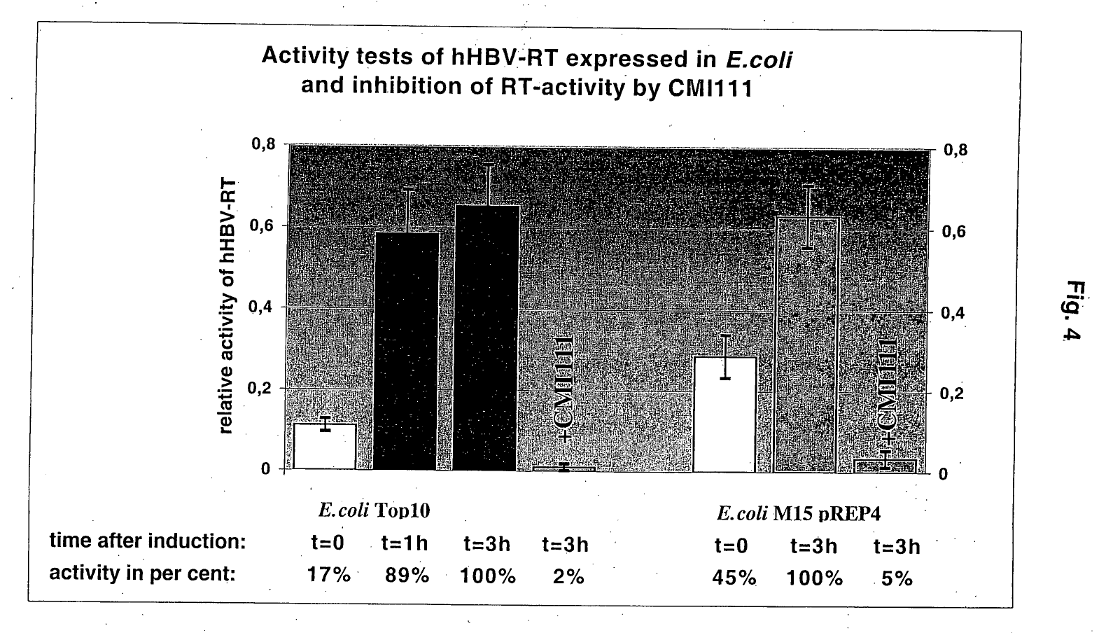

FIG. 4:

RT activity assays are shown. E. coli M15 pREP4 with pQE30::RT and TOP10 with pBAD::RT lysates which express recombinant RT showed distinct activity in the RTA-assay.

The activity of the hHBV-RT expressed in E. coli M15 pREP4 and TOP10 increased remarkably 1 h after induction with IPTG and arabinose, respectively.

Three hours after induction the RT-activity was increased only slightly compared to the activity measured 1 h after induction.

The addition of the inhibitor CMI111 (0,14 mg/ml) to the lysates resulted in a reduction of RT activity down to less than 5% of the original activity.

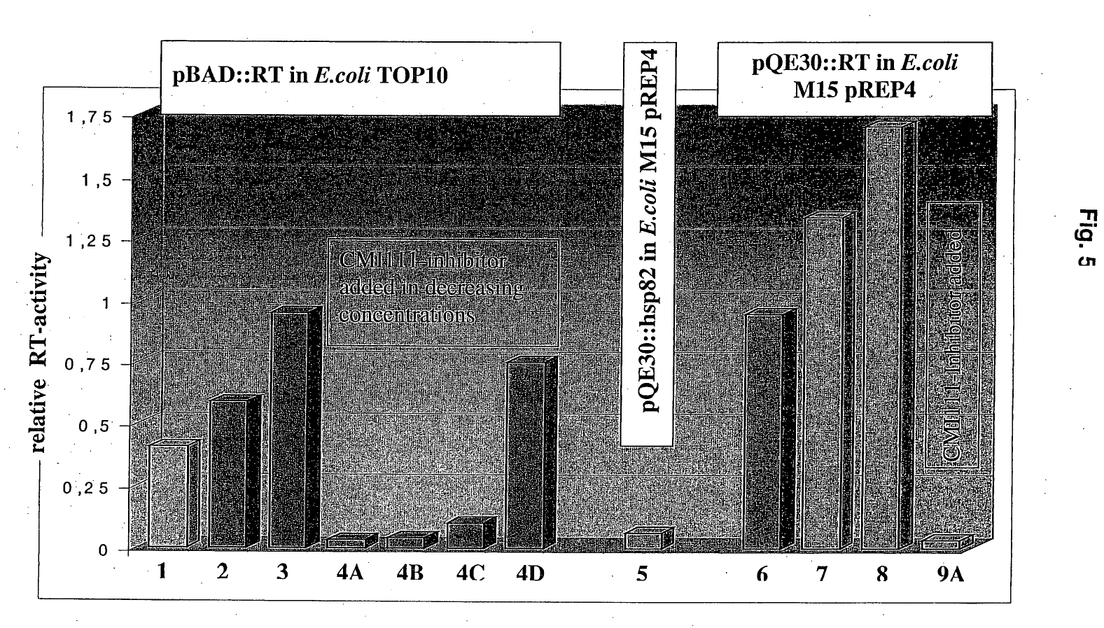

FIG. 5:

E. coli cell lysates were tested for RT activity. E. coli M15 pREP4 with pQE30::hsp82 was used as a negative control. The lysate without recombinantly expressed RT showed marginal RT-activity. This basic activity is due to the intrinsic DNA-polymerases present in E. coli, which are able to synthesise DNA strands on the RNA template. This background activity is about 3% to 10% of the RT activity in lysates from cells expressing recombinant RT. Different concentrations of the RT-inhibitor CMI111 were added and inhibition of recombinant expressed hHBV-RT was observed as dose-dependent. Recombinant RT was expressed in E. coli TOP10 with pBAD::RT and M15 pREP4 with pQE30::RT. The ordinate shows the relative activity of the extracts in the RTA (Reverse Transcriptase Assay). Lane 1 to 4: pBAD::RT in E. coli TOP10; [1] before induction; [2] 1 h after Induction with 0.1% arabinose; [3] 2 h after induction with 0.1% arabinose; [4A] Lane 3 with 0.36 mg/ml CMI111 added; [4B] Lane 3 with 0,09 mg/ml CMI111 added; [4C] Lane 3 with 0,022 mg/ml CMI111 added; [4D] Lane 3 with 0,006 mg/ml CMI111 added. Lane 5: E. coli M15 pREP4 with pQE30::hsp82. Lane 6 to 9: E. coli M15 pREP4 with pQE30::RT; [6] before induction; [7] 1 h after induction with 1 mM IPTG; [8] 2 h after induction with 1 mM IPTG; [9A] Lane 8 with 0,36 mg/ml CMI111 added.

The examples illustrate the invention:

### EXAMPLE 1

### Construction of Vectors for Expression of Reverse Transcriptase of hHBV in E. coli

NcoI and HindIII restriction sites available in the pQE30 vector (Qiagen, Hilden, Germany) were used for cloning. The RT was amplified from a pUC-based template using the primers ON-hHBV-Rcal (AAA ATC ATG ACC CTA TCT TAT CAA CAC TTC CGG) and ON-hHBV-HindIII (AAA AAA GCT TTC ACG GTG GTC TCC ATG CAA CGT GC). The PCR product was digested with the restriction enzymes Rcal and HindIII and cloned into pQE30 (N-terminal 6×His-tag).

For cloning of the RT into the pBAD vector (Invitrogen, Groningen, NL) the restriction sites NcoI and XhoI were used. The RT was amplified with the primers ON-hHBV-Rcal. (AAA ATC ATG ACC CTA TCT TAT CAA CAC TTC CGG) and ON-RT3-new (GAT CCT CGA GCG GTG GTC TCC ATG CAA CG). The PCR product was digested with the restriction enzymes Rcal and HindIII and cloned into pBAD (C-terminal 6×His-tag).

For cloning of RT into pIVEX2.4b (Roche, Basel, CH) the restriction sites NdeI and XhoI were used. The RT was amplified using the primers ON-RT1-new (GAT CCA TAT GCC CCT ATC TTA TCA ACA CTT CCG G) and ON-RT2-new (GAT CCT CGA GTC ATC ACG GTG GTC TCC ATG CAA CG). The PCR product was digested with the restriction sites NdeI and XhoI and cloned into pIVEX2.4b (N-terminal 6×His-tag). Using the same PCR-product, hHBV-RT was also cloned into pIVEX2.2b (NdeI and XhoI).

Enzymes were purchased from Roche (Basel, CH).

The results demonstrate that the following constructs have been cloned successfully:

RT from hHBV cloned into pIVEX-T7-system for in-vitro experiments with N terminal Streptavidin-tag.

RT from hHBV cloned into pIVEX-T7-System with N-terminal His-tag.

RT from hHBV in pQE-T5-system with N-terminal His-tag.

RT from hHBV in pBAD-system with C-terminal His-tag.

### EXAMPLE 2

### Detection of hHBV-RT Expressed in E. coli (Western Blot)

pIVEX2.4b::RT and pBAD::RT expression constructs were propagated and expressed in E. coli BL21DE3 and TOP10, respectively. For detection of recombinant hHBV-RT a mixture of three different monoclonal antibodies were used.

For expression of RT in E. coli cells were induced at O.D.˜0.7. M15 pREP4 cells were induced with 1 mM IPTG, TOP10 cells with 0.1% Arabinose.

Samples were taken at time points t=0 h, 1 h, 2 h, 3 h, 4 h und 5 h after induction. Whole cell samples were separated on SDS-Page (10% polyacrylamid). The gel was finally blotted and the RT detected using αRT-antibodies.

pBAD, pIVEX and pQE30::hsp82 were used as negative controls. For propagation and expression LB-medium was used without exception.

### EXAMPLE 3

### Purification of hHBV-RT by Affinity Chromatography

Purification from E. coli cells:

For purification of pQE30::RT (N-terminal His-tag) strain NiNTA matrix (Qiagen, Hilden, Germany) was used with the following buffers:

Lysis buffer (100 mM NaPO₄ pH8, 300 mM KCl, 5 mM Imidazol);

Wash buffer (100 mM NaPO4 pH8, 300 mM KCl, 20 mM Imidazol);

Elution buffer (100 mM NaPO4 pH8, 300 mM KCl, 5 mM Imidazol).

For preparation of inclusion bodies and subsequent purification 8M urea was added to the buffers described above.

For expression in E. coli M15 pREP cells were induced with 1 mM IPTG at O.D.˜0.6. Then, cells were harvested 5 h after induction, lysed (sonication, French Press) in Lysis buffer and the lysate was centrifuged (Beckman JA25.50, 19000 rpm, 60 min, 4° C.).

The supernatant was added to NiNTA-columns. Fractions of the cell lysate, wash fractions and eluted fractions were separated by SDS-Page (10% polyacrylamid) and subsequently blotted on nitrocellulose-membranes.

Proteins from the cell pellet obtained after centrifugation of sonicated cells were extracted using Lysis buffer with 8M urea. The solubilized fraction was added onto NiNTA-column. Fractions of cell pellet, wash fractions and eluted fractions were treated as described for cytoplasmic fractions. (Test of membrane fractions and inclusion bodies for RT presence).

Purification from in vitro transcription-translation system:

The in vitro transcription and translation reactions were carried out in the RTS₅₀₀-system (Roche) with the appropriate RTS-Kit (Roche) according to the manufacturer's instructions. All reactions were carried out at 30° C., 120 rpm stirring speed for 24 h.

Prior to purification all samples resulting from the in vitro transcription and translation reaction were centrifuged for 10 min at 18 000 rpm, to remove aggregates. Pellets were stored for further analysis. In the case of Strep-tagged RT, samples were loaded on a 0.6 ml StrepTactine Sepharose column, pre-equilibrated with equilibration buffer (100 mM Tris/HCl, 1 mM EDTA, pH 8.0) by gravity flow. The column was washed with 6 ml of equilibration buffer and the RT was eluted with 5 ml of elution buffer (100 mM Tris/HCl, 1 mM EDTA, 2.5 mM desthiobiotin, pH 8.0). During the whole procedure 1 ml samples were collected. The column was regenerated for further use with 10 ml regeneration buffer (100 mM Tris/HCl, 1 mM EDTA, 1 mM HABA, pH 8.0). His-tagged RT RTS-samples were diluted 1:10 with Ni-NTA equilibration buffer (100 mM Na₂HPO₄, 300 mM NaCl, pH 7.4) prior to loading on a 1 ml Ni-NTA-superflow (Qiagen) column under a continuous flow of 1 ml/min. The column was washed with 5 ml of the same buffer and with 5 ml of washing buffer (100 mM Na₂HPO₄, 300 mM NaCl, 30 mM Imidazol, pH 6.8). CS was eluted with 5 ml of elution buffer (100 mM Na₂HPO₄, 300 mM NaCl, 500 mM Imidazol, pH 7.5). 1 ml samples were taken during the whole procedure. Samples were further analysed by SDS-PAGE.

In summary, RT can be expressed and purified using E. coli cells or an in-vitro translation system.

### EXAMPLE 4

### Reverse Transcriptase (RT) Activity Assay and Inhibition by CMI111

RetroTech RTA (Reverse Transcriptase Assay)-kit (Unterschleissheim, Germany) to test RT-activity. Samples from E. coli RT-expression-cultures were analysed according to manufacturers instructions.

Lysates from E. coli M15 pREP4 with pQE30::RT and TOP10 pBAD::RT were tested. M15 pREP4 with pQE30::hsp82 was used as a negative control for intrinsic E. coli DNA-polymerase activity.

The HIV-RT included in the kit was used as a positive control for RT-activity and standard.

Results of corresponding activity assays are shown in FIG. 4. E. coli M15 pREP4 with pQE30::RT and TOP10 with PBAD::RT lysates which express recombinant RT showed distinct activity in the RTA assay.

The activity of the hHBV-RT expressed in E. coli M15 pREP4 and TOP10 increased remarkably 1 h after induction with IPTG and arabinose, respectively.

Three hours after induction the RT-activity was increased only slightly compared to the activity measured 1 h after induction.

The addition of the inhibitor CMI111 (0,14 mg/ml) to the lysates resulted in a reduction of RT activity down to less than 5% of the original activity (FIG. 4).

In FIG. 5, a second set of RT assays is summarised. E. coli cell lysates were tested for RT activity. E. coli M15 pREP4 with pQE30::hsp82 was used as a negative control. The lysate without recombinantly expressed RT showed marginal RT-activity. This basic activity is due to the intrinsic DNA-polymerases present in E. coli, which are able to synthesise DNA strands on the RNA template. This background activity is about 3% to 10% of the RT activity in lysates from cells expressing recombinant RT. Different concentrations of the RT-inhibitor CMI111 were added and inhibition of recombinant expressed hHBV-RT was observed as dose-dependent.

Results presented here indicate that recombinantly expressed RT from hHBV or expressed by using an in-vitro transcription/translation system is functional in the RT-activity assay (Retro-Tech). Furthermore, evidence is provided that the inhibitor CMI111 inhibits RT activity by more than 95%.

### REFERENCE LIST

Ayola, B., P. Kanda, and R. E. Lanford. 1993. High level expression and phosphorylation of hepatitis B virus polymerase in insect cells with recombinant baculoviruses. Virology 194:370-373.

Bartenschlager, R. and H. Schaller. 1988. The amino-terminal domain of the hepadnaviral P-gene encodes the terminal protein (genome-linked protein) believed to prime reverse transcription. EMBO J. 7:4185-4192.

Chang, L. J., P. Pryciak, D. Ganem, and H. E. Varmus. 1989. Biosynthesis of the reverse transcriptase of hepatitis B viruses involves de novo translational initiation not ribosomal frameshifting. Nature 337:364-368.

Chang, L. J., R. C. Hirsch, D. Ganem, and H. E. Varmus. 1990. Effects of insertional and point mutations on the functions of the duck hepatitis B virus polymerase. J Virol 64:5553-5558.

Cho, G., S. G. Park, and G. Jung. 2000. Localization of HSP90 binding sites in the human hepatitis B virus polymerase. Biochem Biophys Res Commun 269:191-196.

Dalman, F. C., E. H. Bresnick, P. D. Patel, G. H. Perdew, S. J. J. Watson, and W. B. Pratt. 1989. Direct evidence that the glucocorticoid receptor binds to hsp90 at or near the termination of receptor translation in vitro. J Biol Chem 264:19815-19821.

Franke, E. K., H. E. Yuan, and J. Luban. 1994. Specific incorporation of cyclophilin A into HIV-1 virions [see comments]. Nature 372:359-362.

Friedman, D. I., E. R. Olson, C. Georgopoulos, K. Tilly, I. Herskowitz, and F. Banuett. 1984. Interactions of bacteriophage and host macromolecules in the growth of bacteriophage lambda. Microbiol Rev 48:299-325.

Gabbara, S. and J. A. Peliska. 1996. Catalytic activities associated with retroviral and viral polymerases. Methods Enzymol 275:276-310.

Hirsch, R. C., J. E. Lavine, L. J. Chang, H. E. Varmus, and D. Ganem. 1990. Polymerase gene products of hepatitis B viruses are required for genomic RNA packaging as wel as for reverse transcription. Nature 344:552-555.

Hu, J. and C. Seeger. 1996a. Expression and characterization of hepadnavirus reverse transcriptases. Methods Enzymol 275:195-208.

Hu, J. and C. Seeger. 1996b. Hsp90 is required for the activity of a hepatitis B virus reverse transcriptase. Proc Natl Acad Sci USA 93:1060-1064.

Hu, J., D. O. Toft, and C. Seeger. 1997. Hepadnavirus assembly and reverse transcription require a multi-component chaperone complex which is incorporated into nucleocapsids. EMBO J. 16:59-68.

Kaplan, P. M., R. L. Greenman, J. L. Gerin, R. H. Purcell, and W. S. Robinson. 1973. DNA polymerase associated with human hepatitis B antigen. J Virol 12:995-1005.

Ladner, S. K., M. J. Otto, ° C. S. Barker, K. Zaifert, G. H. Wang, J. T. Guo, C. Seeger, and R. W. King. 1997. Inducible expression of human hepatitis B virus (HBV) in stably transfected hepatoblastoma cells: a novel system for screening potential inhibitors of HBV replication. Antimicrob Agents Chemother 41:1715-1720.

Lanford, R. E., L. Notvall, and B. Beames. 1995. Nucleotide priming and reverse transcriptase activity of hepatitis B virus polymerase expressed in insect cells. J Virol 69:4431-4439.

Lanford, R. E., L. Notvall, H. Lee, and B. Beames. 1997. Transcomplementation of nucleotide priming and reverse transcription between independently expressed TP and RT domains of the hepatitis B virus reverse transcriptase. J Virol 71:2996-3004.

Nassal, M. 1999. Hepatitis B virus replication: novel roles for virus-host interactions. Intervirology 42:100-116.

Pollack, J. R. and D. Ganem. 1994. Site-specific RNA binding by a hepatitis B virus reverse transcriptase initiates two distinct reactions: RNA packaging and DNA synthesis. J Virol 68:5579-5587.

Qadri, I. and A. Siddiqui. 1999. Expression of hepatitis B virus polymerase in Ty1-his3AI retroelement of Saccharomyces cerevisiae. J Biol Chem 274:31359-31365.

Radziwill, G., H. Zentgraf, H. Schaller, and V. Bosch. 1988. The duck hepatitis B virus DNA polymerase is tightly associated with the viral core structure and unable to switch to an exogenous template. Virology 163:123-132.

Radziwill, G., W. Tucker, and H. Schaller. 1990. Mutational analysis of the hepatitis B virus P gene product: domain structure and RNase H activity. J Virol 64:613-620.

Schlicht, H. J., G. Radziwill, and H. Schaller. 1989. Synthesis and encapsidation of duck hepatitis B virus reverse transcriptase do not require formation of core-polymerase fusion proteins. Cell 56:85-92.

Seifer, M. and D. N. Standring. 1993. Recombinant human hepatitis B virus reverse transcriptase is active in the absence of the nucleocapsid or the viral replication origin, DR1. J Virol 67:4513-4520.

Summers, J. and W. S. Mason. 1982. Replication of the genome of a hepatitis B-like virus by reverse transcription of an RNA intermediate. Cell 29:403-415.

Tavis, J. E. and D. Ganem. 1993. Expression of functional hepatitis B virus polymerase in yeast reveals it to be the sole viral protein required for correct initiation of reverse transcription. Proc Natl Acad Sci USA 90:4107-4111.

Tavis, J. E. and D. Ganem. 1996. Evidence for activation of the hepatitis B virus polymerase by binding of its RNA template. J Virol 70:5741-5750.

Tavis, J. E., B. Massey, and Y. Gong. 1998. The duck hepatitis B virus polymerase is activated by its RNA packaging signal, epsilon. J Virol 72:5789-5796.

Tavis, J. E., S. Perri, and D. Ganem. 1994. Hepadnavirus reverse transcription initiates within the stem-loop of the RNA packaging signal and employs a novel strand transfer. J Virol 68:3536-3543.

Thali, M., A. Bukovsky, E. Kondo, B. Rosenwirth, C. T. Walsh, J. Sodroski, and H. G. Gottlinger. 1994. Functional association of cyclophilin A with HIV-1 virions [see comments]. Nature 372:363-365.

Wang, G. H. and C. Seeger. 1992. The reverse transcriptase of hepatitis B virus acts as a protein primer for viral DNA synthesis. Cell 71:663-670.

Wang, G. H., F. Zoulim, E. H. Leber, J. Kitson, and C. Seeger. 1994. Role of RNA in enzymatic activity of the reverse transcriptase of hepatitis B viruses. J Virol 68:8437-8442.

Zoulim, F. and C. Seeger. 1994. Reverse transcription in hepatitis B viruses is primed by a tyrosine residue of the polymerase. J Virol 68:6-13.

1. A method of producing a functional cell-free hepatitis B virus (HBV) reverse transcriptase (RT) comprising the steps of:
(a) expressing HBV-RT in
(aa) E. coli cells employing a suitable expression vector; or
(ab) a cell-free transcription-translation system
(b) if expression was carried out in E. coli cells, lysing said E. coli cells and
(c) purifying HBV-RT from the E. coli lysate or from said transcription-translation system.

2. The method of claim 1 wherein said HBV-RT is expressed as a fusion protein.

3. The method of claim 2 wherein said fusion protein comprises a tag.

4. The method of claim 3 wherein said tag is fused N-terminally to said HBV-RT.

5. The method of claim 3 wherein said tag is fused C-terminally to said HBV-RT.

6. The method of claim 3 wherein said tag is selected from the group of His-tag, Streptavidin-tag, HA-tag, GST-tag, CBP-tag, MBP-tag and FLAG-tag.

7. The method of claim 1 wherein step (c) comprises the steps of
(ca) centrifugation of the lysate or the transcription-translation system at about 30000×g so as to remove the non-soluble fraction of the lysate or the transcription-translation system; and
(cb) removing the supernatant containing the HBV-RT

8. The method of claim 7 further comprising the step of (cc) purifying the supernatant via chromatography.

9. The method of claim 8 wherein said chromatography is affinity chromatography and wherein said affinity is specific for the tag.

10. The method of claim 1 wherein said HBV-RT is human HBV-RT.

11. A method of screening for an inhibitor of HBV-RT activity comprising the steps of
(a) contacting the cell-free HBV-RT produced according to the method of claim 1 with a potential inhibitor; and
(b) assaying whether said potential inhibitor inhibits HBV-RT activity.

12. The method of claim 11 wherein assaying in step (b) is effective via a readout system.

13. The method of claim 12 wherein said readout system is non-radioactivity-based.

14. The method of claim 11 wherein assaying step (b) is qualitative.

15. The method of claim 11 wherein assaying in step (b) is qualitative.

16. The method of claim 11 wherein said potential inhibitor is a plant produce or a plant-derived product.

17. The method of claim 16 wherein said plant is Phyllantus.

18. The method of claim 17 wherein said Phyllanthus is Phyllantus amarus.

19. A method of refining the inhibitor identified by the method of claim 11 comprising
(a) modeling said inhibitor by peptidomimetics; and
(b) chemically synthesizing the modeled inhibitor.

20. A method of modifying an inhibitor identified by the method of claim 11 as a lead compound to achieve
(i) modified site of action, spectrum of activity, organ specificity, and/or
(ii) improved potency, and/or
(iii) decreased toxicity (improved therapeutic index), and/or
(iv) decreased side effects, and/or
(v) modified onset of therapeutic action, duration of effect, and/or
(vi) modified pharmakinetic parameters (resorption, distribution, metabolism and excretion), and/or
(vii) modified physico-chemical parameters (solubility, hygroscopicity, color, taste, odor, stability, state), and/or
(viii) improved general specificity, organ/tissue specificity, and/or
(ix) optimized application form and route
by
(i) esterification of carboxyl groups, or
(ii) esterification of hydroxyl groups with carbon acids, or
(iii) esterification of hydroxyl groups to, e.g. phosphates, pyrophosphates or sulfates or hemi succinates, or
(iv) formation of pharmaceutically acceptable salts, or
(v) formation of pharmaceutically acceptable complexes, or
(vi) synthesis of pharmaceutically active polymers, or
(vii) introduction of hydrophilic moieties, or
(viii) introduction/exchange of substituents on aromates or side chains, change of substituent pattern, or
(ix) modification by introduction of isosteric or bioisosteric moieties, or
(x) synthesis of homologous compounds, or
(xi) introduction of branched side chains, or
(xii) conversion of alkyl substituents to cyclic analogues, or
(xiii) derivatisation of hydroxyl group to ketales, acetates, or
(xiv) N-acetylation to amides, phenylcarbamates, or
(xv) synthesis of Mannich bases, imines, or
(xvi) transformation of ketones or aldehydes to Schiff's bases, oximes, acetates, ketales, enolesters, oxazolidines, thiozolidines
or combinations thereof.

21. A method of producing a pharmaceutical composition comprising the step of formulating the inhibitor identified by the screening method of claim 11 with a pharmaceutically acceptable carrier or diluent.

22. A method for in vitro synthesis of cDNA using the HBV-RT produced according to the method of claim 1.

23. A method of evaluating the batch to batch consistency of Phyllantus amarus extracts/inhibitor preparations and/or their shelf life, comprising contacting the HBV-RT produced according to the method of claim 1 with said batch and assessing the RT-activity.
